(19) United States
(12) Patent Application Publication
(10) Pub. No.: US 2016/0346349 A1
(43) Pub. Date: Dec. 1, 2016

(54) PREPARATION AND USE OF A PROTEIN-ENRICHED SOLUBLE FIBER COMPOSITION

(71) Applicant: Albert BURGIN, South Daytona, FL (US)

(72) Inventor: Albert BURGIN, South Daytona, FL (US)

(21) Appl. No.: 15/074,291

(22) Filed: Mar. 18, 2016

Related U.S. Application Data
(60) Provisional application No. 62/168,766, filed on May 30, 2015.

(51) Int. Cl. A61K 38/17

(52) U.S. Cl. CPC A61K 38/1709; A61K 38/1703; A23L 1/305; A23L 1/308; A23V 2002/00

(57) ABSTRACT

Methods are described for preparing a protein-enriched soluble fiber composition in granular form, and the use of the product in edible foodstuffs and beverages.

### CROSS REFERENCE TO RELATED APPLICATION

The present application claims the benefit of U.S. Provisional Application Ser. No. 62/168,766, filed May 30, 2015; which is hereby incorporated by reference in its entirety, including any figures, tables, or drawings.

### BACKGROUND OF THE INVENTION

1. Field of the Invention

This invention relates to methods for preparing a protein-enriched soluble fiber composition and the use of this material in making a dietary supplement. 2. Related Art

The soluble fiber useful according to the subject invention is well known to those skilled in this art. Specifically exemplified herein is the use of pectin and guar gum compositions as the soluble fiber source. Pectins are a group of high-molecular-weight heterogenous polysaccharides that serve as fundamental structural components of plant cell walls in fruits and vegetables, particularly limes, lemons, grapefruit, and oranges. Since pectin and guar gum are not susceptible to hydrolysis by the alimentary tract enzymes of humans, it is classified as a water-soluble fiber dietary supplement.

Soluble fiber has been shown to be an effective hypocholesterolemic agent with minimal side effects (Reiser [1987] Food Technol. 31-91). It has also been found that grapefruit pectin inhibits hypercholesterolemia and atherosclerosis in miniature swine (Baekey et al. [1988] Clin. Cardiology 11:595-600). Furthermore, grapefruit pectin has proved to lower plasma cholesterol in human volunteers who were hypercholesterolemic, and to improve the ratio of low density lipoprotein cholesterol (LDLC) to high density lipoprotein cholesterol (HDLC) (Cerda [1988] Clin. Cardiology 11:589-594).

Simple methods that allow the manufacture of soluble fiber food compositions are needed to provide the general public with dietary forms of soluble fiber. The present invention provides a preparation method that achieves this goal.

### BRIEF SUMMARY OF THE INVENTION

The subject invention provides a unique and advantageous method for entrapping a material within a protein matrix. Advantageously, the entrapped material, when eaten, moves through the stomach to the intestine before the protein is broken down, which then releases the entrapped compound.

Specifically exemplified is a simple and efficient procedure to prepare a soluble fiber product entrapped in a protein matrix that can be included in edible foodstuffs or which can be readily homogenized as an additive to drinkable fluids such as beverages.

In a preferred embodiment of the subject invention, the soluble fiber and protein composition is prepared by a process that comprises a compression step that imparts advantageous properties to the final product. Specifically exemplified herein is a process wherein a compression step utilizes an extruder to compress the water soluble fiber/protein composition. The extrusion step is followed by drying, sizing, and, advantageously, further mechanical treatment and heating.

One specific embodiment of the subject invention pertains to a four-step process for producing the soluble fiber product. In a first step, protein and soluble fiber are blended to create a uniform dispersion of these ingredients. Once the uniform dispersion of protein and soluble fiber is created, the dispersion is mixed with water and introduced into an extruder as the second step of the process. A third step involves drying and sizing of the compressed protein-enriched soluble fiber product. A further step of the process provides additional heating and agitation of the composition that allows the protein in the composition to be further denatured.

In the initial mixing step, a range of protein from about 25% to about 60% can be used. The soluble fiber content can range from about 40% to about 75%. Preferably, the composition further comprises about 0% to about 5% oil. After the initial blending, water is added to create a water content of about 40% to about 50%, preferably about 46% to about 48%. In the second step of the process, the extrusion is carried out so that the product leaving the extruder still comprises less than 50% water and, preferably, comprises about 46-48% water. The product is then dried. The water content of the product after drying is preferably less than about 5%, more preferably less than about 3%, and most preferably is about 1-2%.

In a preferred embodiment, the soluble fiber component comprises a majority of guar gum and a smaller component of pectin. Preferred in the method is the use of citrus (e.g., grapefruit) pectin, particularly HM pectin, as the pectin source. Preferred sources of the protein include egg white or skimmed milk. Preferably, all ingredients are dry-based.

A further aspect of the present invention is an edible foodstuff or beverage containing the protein-enriched soluble fiber composition prepared by the aforementioned method. Edible foodstuffs and beverages to which the composition of the subject invention can be added include desserts, salad dressings, gelatin desserts, puddings, carbonated beverages, and juices.

A further aspect of the present invention is a dietary supplement comprising a protein-enriched soluble fiber composition prepared according to the aforementioned method.

### DETAILED DISCLOSURE OF THE INVENTION

The subject invention provides a process for entrapping a material within a protein matrix. In a preferred embodiment, the subject invention concerns a unique process for creating a protein-enriched soluble fiber composition.

The present method for preparing a protein-enriched soluble fiber composition includes an initial step of contacting soluble fiber with a water-soluble, edible protein. Water is added to the mixture. Following this initial contacting step, the composition is subjected to elevated pressures sufficient to bring the protein and soluble fiber into close molecular proximity. Close molecular proximity means that the soluble fiber can be entrapped within the protein matrix when the protein is denatured by a heat treatment. Preferably, the soluble fiber is hydrated by the elevated pressure. The pressure necessary for this step is preferably provided by an extrusion process.

A third step involves drying and sizing of the compressed protein-enriched soluble fiber product. A further step of the process provides additional heating and agitation of the composition that allows the protein in the composition to be further denatured.

The composition produced according to the subject invention has advantageous properties in terms of its taste, consistency, stability, and its physiological properties. The process of the subject invention yields a product rich in soluble fiber, wherein that fiber is entrapped within a protein matrix. The product of the subject invention can be stored for extended periods of time without losing its vital characteristics. As with other soluble fiber products, the product of the subject invention can be used to help reduce cholesterol and triglyceride levels. Furthermore, it has been discovered that the soluble fiber product of the subject invention can be used in conjunction with medications which reduce cholesterol and triglyceride levels. Surprisingly, the use of the instant soluble fiber composition does not reduce or compromise the effects of the cholesterol or triglyceride medication, and, in fact, substantial reductions in cholesterol and triglyceride levels were achieved.

In a preferred embodiment, the protein-enriched soluble fiber compositions of the subject invention can be produced using a four-step process. In a first step, the protein and soluble fiber components are blended to create a uniform, homogenous dispersion. The protein component should be at least about 25% and preferably about 40 to about 45%. The protein component can be, for example, egg albumin, and should not be denatured at the beginning of the process. Those skilled in the art having the benefit of the teachings provided herein would readily recognize that other proteins could be used in the process of the subject invention. Also, a mixture of proteins can be used. The soluble fiber used according to the subject invention can be one or more of such fibers well known to those skilled in the art. For example, guar gum and/or pectin can be used as described herein. The soluble fiber component can be up to about 75% of the composition. Preferably, the soluble fiber component is about 50% to about 60%.

The protein and soluble fiber can be mixed in any appropriate device which will create a uniform, homogenous dispersion. For example, a ribbon blender can be used to slowly tumble the ingredients. The time of blending can be, for example, about 5 minutes to about 20 minutes, or more. In one embodiment of the subject invention, an edible oil is added to the mixture of soluble fiber and protein. The oil can be, for example, canola oil or other such vegetable oil. The amount of oil can be from 0% to about 5%. An oil content of about 3.5% is preferred. The oil can be blended with the protein and soluble fiber. The blending should be of a duration and nature to create an essentially dry, uniform dispersion without lumps.

As an additional aspect of the first step of the process of the subject invention, water is added to the protein and soluble fiber mixture. Water content of the composition should be less than about 55%, preferably less than about 50% and more than about 40%, and, most preferably, between about 46 and about 48%. Once the proper moisture content is achieved, the composition passes on to the second step of the process, which is the extrusion step.

During the extrusion step of the subject invention, the soluble fiber and protein composition is subjected to pressures sufficient to force the protein and soluble fiber into close proximity at the molecular level so that the soluble fiber can become entrapped within a protein matrix upon denaturing of the protein.

Upon exiting the extruder, the soluble fiber and protein composition is subjected to the third stage of the process, which is a drying step. The drying step is preferably carried out at temperatures which are sufficient to denature the protein, in addition to removing water. This temperature may be, for example, above about 300° F. and, preferably, is about 400° F. for about 5-10 minutes. The drying process can continue for additional time at, preferably, lower temperatures in order to reduce the water content to less than about 5%. For example, subjecting the composition to about 15 to about 20 minutes at about 300° F. has been found to reduce the moisture to between about 1% and about 2%.

After drying, the composition of the subject invention can be subjected to standard milling and sizing steps. The mesh used for the sizing step according to the subject invention is preferably smaller than 200 mesh. A mesh size between 200 and 300 is preferred, with about 250±25 particularly preferred.

The resultant composition is then subjected to a final processing step whereby the protein in the composition is further denatured. Advantageously, this step yields a more fluid composition that does not gel as easily over time as the same composition not subjected to this final processing step. This step involves simultaneously heating and agitating the composition.

Agitation as described herein can be accomplished using, for example, a ribbon blender or a screw auger. Other suitable means of agitation recognized by those skilled in the art as capable of achieving the same results may also be used. During the agitation step, the composition is heated to a temperature preferably between about 200° F. and about 300° F. for about 20 to about 30 minutes.

In an alternative embodiment, milling and sizing can take place after the final denaturing step.

Finally, the composition can undergo an agglomeration step with, for example, gum arabic. When the composition is ready for shipping, it can be put, for example, into cans, and the moisture content at that point may be, for example, about 5%.

A suitable protein used in this invention can be any form of a water soluble, edible protein as long as it has not been denatured prior to the start of the process. Preferred protein sources include egg white and skimmed milk. The egg white, skimmed milk, or other protein is preferably dried.

The soluble fiber used as the starting material for the method is preferably guar gum and/or a pectin. Locust bean gum and psyllium, for example, can also be used. The pectin may be a citrus (e.g., grapefruit) HM pectin. Dry base commercially prepared pectins are readily available.

A person skilled in the art having the benefit of this disclosure can appreciate that other materials can be advantageously entrapped in the protein matrix.

The method of heating can be, for example, electric or steam. Those skilled in the art, however, will recognize that other forms of heating are also suitable for supplying the elevated temperatures described herein.

Following are examples which illustrate procedures for practicing the invention. These examples should not be construed as limiting. All percentages are by weight and all solvent mixture proportions are by volume unless otherwise noted.

### EXAMPLE 1—SPECIFIC SOLUBLE FIBER COMPOSITION

In one specific embodiment, the composition of the subject invention may have the following formula:

| | |
|---|---|
| Egg white | 43.5% |
| Guar gum | 41.7% |
| Citrus pectin | 11.3% |
| Canola oil | 3.5% |

A typical serving may contain approximately 12 grams of materials. Of this 12 grams, there are about 5 grams protein and 5 grams of soluble fiber with the balance being primarily oil and water.

### EXAMPLE 2—FOOD PRODUCTS USING PROTEIN-ENRICHED SOLUBLE FIBER COMPOSITION

The protein-enriched soluble fiber composition prepared by the described method can be included in various foodstuffs and used as a dietary supplement. Typical products in which the soluble fiber composition finds use includes jams, marmalades, jellies, preserves, and fruit butters; in frozen food compositions such as ice cream and sherbet; in baked goods such as cookies, pastries, and other foodstuff containing wheat and other flour; in beverages such as juice; in toppings, sauces, and puddings; and in salad dressings. In liquid foods and beverages, the composition will typically be an additive, whereas the product can be directly incorporated into dry products during the manufacturing process. Dry products can include cereals and oatmeal.

The protein-enriched soluble fiber composition of the present invention is not only nutritious, because of high protein concentrations, but also is useful as a dietary supplement which is effective in lowering plasma cholesterol. Wide application of the soluble fiber product of the subject invention in foods is also possible without impairing the physical properties and the tastes of the foods.

Orange juice, using the products of this invention, can be prepared. The product of Example 1 can be dissolved in orange juice in a ratio of 11.8 g product/250 ml juice. The product readily disperses in the juice, and there is no change in the hedonic characteristics of the orange juice.

The products of Example 1 can also be added to fruit bars, toffee bars, and cold cereal. The products can be seasoned and used as croutons on tossed salad.

### EXAMPLE 3—USE OF PROTEIN-ENRICHED SOLUBLE FIBER COMPOSITIONS WITH MEDICATIONS

The compositions of the subject invention are particularly advantageous because they can be used in conjunction (sequentially or simultaneously) with prescription medications for lowering cholesterol or triglyceride levels. Such medicines include, but are not limited to, MEVACOR, LESCOL, ZOCAR, and LOPID. Advantageously, these medications, when used together with the composition of the subject invention, result in substantial decreases in levels of total cholesterol and triglycerides.

The invention now being fully described, it will be apparent to one of ordinary skill in the art that various changes and modifications can be made without departing from the spirit or scope of the invention.

I claim:

1. In a method for entrapping a desired material within a protein matrix, wherein said method comprises the following steps:
(a) said material is first mixed with undenatured protein to create a uniform dispersion;
(b) the dispersion created in step (a) is then mixed with water and subjected to elevated pressures sufficient to force said protein and said material into close molecular proximity; and
(c) the dispersion is then subjected to heat sufficient to denature the protein, thereby entrapping the desired material within the protein matrix,
an improvement comprising a further heating step whereby the protein in the composition is further denatured.

2. The method, according to claim 1, wherein said desired material is a soluble fiber compound.

3. The method, according to claim 2, wherein said soluble fiber compound is selected from the group consisting of guar gum and pectins, and the protein is selected from the group consisting of egg white and skimmed milk.

4. The method, according to claim 1, wherein said pressure is applied by an extrusion process.

5. The method, according to claim 1, wherein the dispersion created in step (a) comprises protein at a concentration of about 25% to about 60% and soluble fiber at a concentration of about 40% to about 75%.

6. The method, according to claim 5, wherein said composition further comprises up to about 5% edible oil.

7. The method, according to claim 6, wherein said oil is a vegetable oil.

8. The method, according to claim 4, wherein the composition exiting the extrusion step is heated to at least about 300° F. for at least about 5 minutes.

9. The method, according to claim 8, wherein the composition exiting the extrusion step is heated to about 400° F. for about 5 to about 10 minutes followed by heating at about 300° F. for about 15 to about 20 minutes.

10. The method, according to claim 1, wherein said heating process reduces the water content of the composition to less than about 5%.

11. The method, according to claim 1, which further comprises a sizing step wherein particles smaller than about 200-mesh are created.

12. The method, according to claim 1, wherein, in the further heating step, the composition is heated to at least about 200° F. for at least about 20 minutes.

13. The method, according to claim 13, wherein the composition is heated to between about 200° F. and about 300° F. for about 20 to about 30 minutes.

14. The method, according to claim 1, wherein, during the final heating step, the composition is agitated while being heated.

15. A composition comprising a material entrapped within a protein matrix wherein said material entrapped within a protein matrix has the characteristics of that which is produced by the method of claim 1.

16. A method for reducing cholesterol or triglyceride levels in a human or animal wherein said method comprises administering to said human or animal an effective amount of a composition of claim 1.
